(19) United States
(12) Patent Application Publication
(10) Pub. No.: US 2023/0037994 A1
(43) Pub. Date: Feb. 9, 2023

(54) LEARNING DEVICE, LEARNING METHOD, AND MEASUREMENT DEVICE

(71) Applicant: KABUSHIKI KAISHA TOKAI RIKA DENKI SEISAKUSHO, Aichi (JP)

(72) Inventors: Ryugo FUJITA, Aichi (JP); Daisuke KAWAMURA, Aichi (JP); Yuuki NAWA, Aichi (JP); Minoru OTAKE, Aichi (JP); Tetsuya HIROTA, Aichi (JP)

(73) Assignee: KABUSHIKI KAISHA TOKAI RIKA DENKI SEISAKUSHO, Aichi (JP)

(21) Appl. No.: 17/768,386

(22) PCT Filed: Aug. 11, 2020
(86) PCT No.: PCT/JP2020/030609

§ 371 Date: Apr. 12, 2022

(30) Foreign Application Priority Data
Nov. 18, 2019 (JP) 2019-208016

(51) Int. Cl. A61B 5/352; G06N 20/00

(52) U.S. Cl. CPC A61B 5/352; G06N 20/00; A61B 2503/22

(57) ABSTRACT

There is provided a learning device, including a learning unit that learns output related to a target feature point to be observed in a repetition section observed periodically, with the use of the first sensor data being acquired by the first system and having a time length corresponding to the repetition section, as learning data, and of teacher data based on the second sensor data acquired by the second system at a time point when a specific period of time has elapsed since a start time point of the time length related to the first sensor data, the second system being less affected by noises than the first system, in which the specific period of time is set on the basis of a time length from a start time point of the repetition section to a time point at which the target feature point is expected to appear.

### TECHNICAL FIELD

The present invention relates to a learning device, a learning method, and a measurement device.

### BACKGROUND ART

Recently, there have been developed the technique of performing discrimination, estimation, and the like using the machine learning technique. For example, Patent Literature 1 discloses the technique related to the learning for predicting a future value of time series data. With the technique, it is possible to predict which value is shown at an arbitrary time point in the future in data acquired in time series.

### CITATION LIST

### Patent Literature

Patent Literature 1: JP 2001-325582A

### SUMMARY OF INVENTION

### Technical Problem

However, the technique described in Patent Literature 1 uses a future value at a predicted time, as teacher data for learning. In this case, it is difficult to perform learning that reflects the features of a transition of time series data after the future value, and the like.

In view of the above-described aspects, the present invention aims at providing a mechanism that enables further effective learning of the relation between feature points in time series data.

### Solution to Problem

In order to solve the above-described problem, one aspect of the present invention provides a learning device, including a learning unit that learns output related to a target feature point to be observed in a repetition section observed periodically along a progress of time, with the use of first sensor data being acquired by a first system and having a time length corresponding to the repetition section, as learning data, and of teacher data based on second sensor data acquired by a second system at a time point when a specific period of time has elapsed since a start time point of the time length related to the first sensor data, the second system being less affected by noises than the first system, in which the specific period of time is set on the basis of a time length from a start time point of the repetition section to a time point at which the target feature point is expected to appear.

Further, in order to solve the above-described problem, another aspect of the present invention provides a learning method, including learning output related to a target feature point to be observed in a repetition section observed periodically along a progress of time, with the use of first sensor data being acquired by a first system and having a time length corresponding to the repetition section, as learning data, and of teacher data based on second sensor data acquired by a second system at a time point when a specific period of time has elapsed since a start time point of the time length related to the first sensor data, the second system being less affected by noises than the first system, in which the specific period of time is set on the basis of a time length from a start time point of the repetition section to a time point at which the target feature point is expected to appear.

Further, in order to solve the above-described problem, another aspect of the present invention provides a measurement device, including a measurement unit that performs measurement related to a target feature point to be observed in first sensor data, with the first sensor data acquired by a first system as an input, in which the measurement unit performs measurement related to the target feature point using a learned model constructed by learning output related to the target feature point in a repetition section observed periodically along a progress of time with the use of the first sensor data having a time length corresponding to the repetition section, as learning data, and of teacher data based on second sensor data acquired by a second system at a time point when a specific period of time has elapsed since a start time point of the time length related to the first sensor data, the second system being less affected by noises than the first system, and the specific period of time is set on the basis of a time length from a start time point of the repetition section to a time point at which the target feature point is expected to appear.

### Advantageous Effects of Invention

As described above, the present invention provides a mechanism that enables further effective learning of the relation between feature points in time series data.

### BRIEF DESCRIPTION OF DRAWINGS

FIG. 1 is a diagram illustrating a functional configuration example of a learning device 10 according to an embodiment of the present invention.

FIG. 2 is a diagram illustrating a functional configuration example of a measurement device 20 according to the embodiment.

FIG. 3 is a diagram illustrating an example of a general electrocardiographic waveform in a single cycle.

FIG. 4 is a diagram illustrating a correspondence example between learning data and teacher data according to an embodiment of the present invention.

FIG. 5 is a diagram illustrating an image of the measurement of a target feature point by a measurement unit 220 according to the embodiment.

FIG. 6 is a diagram illustrating an image of the measurement of a target feature point by the measurement unit 220 according to the embodiment.

FIG. 7 is a diagram illustrating the accuracy of detection of an R wave using a learned model according to the embodiment.

FIG. 8 is a flow chart illustrating a flow of a learning phase according to the embodiment.

FIG. 9 is a flow chart illustrating a flow of a measurement phase according to the embodiment.

### DESCRIPTION OF EMBODIMENTS

Hereinafter, referring to the appended drawings, preferred embodiments of the present invention will be described in detail. It should be noted that, in this specification and the drawings, structural elements that have substantially the same function and structure are denoted with the same reference signs, and repeated explanation thereof is omitted.

### Configuration Example

(Learning Device 10)

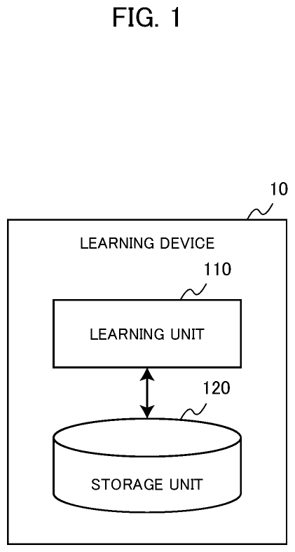

A learning device 10 of the embodiment may be a device that performs supervised learning with the use of, as an input, the same kind of sensor data acquired in synchronization in the time axis by two different systems. Here, the supervised learning indicates a method in which sets of input data (learning data) and correct answer data (teacher data) corresponding to the input data are provided to a computer so that the computer learns the correspondence therebetween. FIG. 1 is a diagram illustrating a functional configuration example of the learning device 10 according to the embodiment. As illustrated in FIG. 1, the learning device 10 of the embodiment may include a learning unit 110 and a storage unit 120.

The learning unit 110 of the embodiment is characterized in learning the output related to a target feature point to be observed in a repetition section observed periodically along the progress of time with the use of the first sensor data being acquired by the first system and having a time length corresponding to the repetition section as learning data and of teacher data based on the second sensor data acquired by the second system at a time point when a specific period of time has elapsed since the start time point of the time length related to the first sensor data, the second system being less affected by noises than the first system. Moreover, the above-described specific period of time may be set on the basis of the time length from the start time point of the repetition section to a time point at which a target feature point is expected to appear. With this configuration, it is possible to learn the features of a data transition after the above-described target feature point, and the like, and construct a higher-accuracy learned model.

The learning unit 110 of the embodiment may perform the above-described learning using an arbitrary machine learning method capable of achieving supervised learning. The learning unit 110 performs learning using an algorithm such as a neutral network or a support vector machine (SVM), for example.

The functions of the learning unit 110 are achieved by a processor such as a graphics processing unit (GPU), for example. The details of the functions of the learning unit 110 according to the embodiment will be specifically described separately.

The storage unit 120 of the embodiment stores various kinds of information related to operations of the learning device 10. The storage unit 120 stores, for example, the first sensor data and the second sensor data, various kinds of parameters, and the like that are used in learning by the learning unit 110.

The above has described the functional configuration example of the learning device 10 according to the embodiment. Note that the configuration described above using FIG. 1 is merely an example and the configuration of the learning device 10 of the embodiment is not limited thereto. The learning device 10 of the embodiment may further include, for example, an operation unit that receives operations by an operator, an output unit that outputs various kinds of data, and the like. The configuration of the learning device 10 of the embodiment can be modified flexibly depending on specifications and uses.

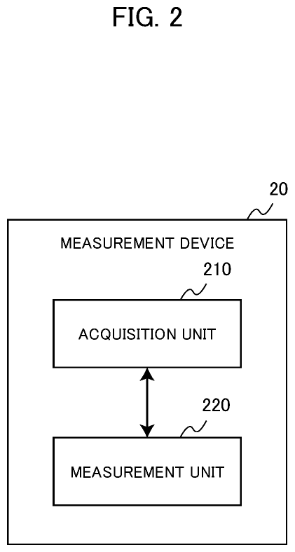

The following will describe a functional configuration example of the measurement device 20 according to the embodiment. The measurement device 20 of the embodiment may be a device that performs measurement related to a target feature point to be observed in sensor data acquired along the progress of time using a learned model constructed by the learning device 10. FIG. 2 is a diagram illustrating a functional configuration example of the measurement device 20 according to the embodiment. As illustrated in FIG. 2, the measurement device 20 of the embodiment may include an acquisition unit 210 and a measurement unit 220.

The acquisition unit 210 of the embodiment is a component for acquiring the first sensor data along the progress of time. For this reason, the acquisition unit 210 of the embodiment includes various kinds of sensors in accordance with the characteristics of the first sensor data to be acquired.

The measurement unit 220 of the embodiment performs measurement related to a target feature point to be observed in the first sensor data, with the first sensor data acquired by the acquisition unit 210 as an input. Here, the measurement unit 220 of the embodiment performs output related to the target feature point using a learned model constructed by learning by the learning unit 110. That is, the measurement unit 220 of the embodiment is characterized in performing measurement related to a target feature point using a learned model constructed by learning the output related to the target feature point in a repetition section observed periodically along the progress of time with the use of the first sensor data having a time length corresponding to the repetition section as learning data and of teacher data based on the second sensor data acquired at a time point when a specific period of time has elapsed since the start time point of the time length related to the first sensor data.

With the above-described configuration, it is possible to efficiently remove the influence by noises from the first sensor data and perform measurement related to a target feature point with high accuracy. Note that the functions of the measurement unit 220 of the embodiment are achieved by various processors.

The above has described the functional configuration example of the measurement device 20 according to the embodiment. Note that the configuration described above using FIG. 2 is a mere example and the functional configuration of the measurement device 20 of the embodiment is not limited thereto. The measurement device 20 of the embodiment may further include an operation unit, an output unit, an analysis unit that performs analysis related to a measured target feature point, a notification unit that performs various kinds of notifications on the basis of analysis results, and the like. The configuration of the measurement device 20 of the embodiment may be modified flexibly in accordance with the characteristics of a target feature point to be measured, uses and utilizations, and the like.

<Details>

The following will describe sensor data of the embodiment using concrete examples. Recently, there have been developed devices that acquire various kinds of sensor data. Moreover, even in the case of acquiring the same kind of sensor data, a plurality of systems may exist. The above-described sensor data may include vital data indicating life signs of a subject. Here, it is assumed that the change in voltage caused by the cardiac activity of a subject is acquired as an electrocardiographic waveform, as an example of the vital data.

The system of acquiring an electrocardiographic waveform may be, for example, a system of a three-point inductive method or a 12 inductive method in which a plurality of electrodes are attached directly on the skin of a subject so that the change in voltage is recorded with the electrodes. With such a system, it is possible to acquire a high-accuracy electrocardiographic waveform less affected by noises. However, such a system may often limit activities of a subject, or may cause a subject to feel annoyed because the electrodes are attached directly on the skin.

Moreover, another system for acquiring an electrocardiographic waveform may be a system in which with electrodes provided at a plurality of positions to be assumedly in contact with a subject, a change in voltage acquired when the subject comes into contact with the electrodes is recorded. Such a system is used to acquire an electrocardiographic waveform of a subject operating a device, for example. As an example, there is known a technique of acquiring an electrocardiogram of a driver driving a mobile body such as a vehicle using electrodes provided at a steering or a driver's seat with which the driver assumedly comes into contact during driving. With such a technique, it is not necessary to attach electrodes directly onto the skin of the driver, whereby an electrocardiographic waveform can be acquired without requiring driver's consciousness. In such a case, meanwhile, noises easily occur due to the movement of a driver's body caused by driving action, vibrations of a vehicle, and the like, which may deteriorate the accuracy of an acquired electrocardiographic waveform.

As described above, each of a plurality of systems for acquiring sensor data has an advantage, while there may exist a case where the accuracy of acquired sensor data varies. Therefore, there has been demanded a technique of improving the acquisition accuracy of sensor data while making use of the advantage of a certain system.

To solve the above-described aspect, the learning unit 110 of the embodiment performs learning with the use of the first sensor data acquired by the first system as learning and of teacher data based on the second sensor data acquired by the second system in synchronization in the time axis with the first sensor data, the second system being less affected by noises than the first system. In this manner, it is possible to efficiently remove the influence by noises from the first sensor data and perform measurement related to a target feature point with high accuracy.

Meanwhile, in a case where the second sensor data corresponding to the end of the time length of the first sensor data or the second sensor data acquired after the end thereof is used here as teacher data, it is difficult to enable the learning unit 110 to learn the information of a data transition after such teacher data, and the like.

In view of the above-described aspect, the learning unit 110 of the embodiment may use, as learning data, the first sensor data having a time length corresponding to a repetition section observed periodically along the progress of time. Moreover, the learning unit 110 of the embodiment may perform learning with the use of teacher data based on the second sensor data acquired at a time point when a specific period of time has elapsed since the start time point of the above-described time length. Here, the above-described specific period of time may be set on the basis of the time length from the start time point of the repetition section to a time point at which a target feature point is expected to appear. In this manner, it is possible to perform learning using the information before and after the teacher data and thus construct a high-accuracy learned model.

Furthermore, the above-described repetition section may include at least another feature point having regularity, regarding the appearance, in the time axis with a target feature point. In this case, it is possible to construct a learned model enabling higher accuracy measurement related to a target feature point by learning the regularity in the time axis between the target feature point and another feature point in the repetition section.

The following will describe, as an example, the case where each of the first sensor data and the second sensor data of the embodiment is an electrocardiographic waveform recording the cardiac activity of a subject. That is, the first sensor data of the embodiment may be a first electrocardiographic waveform acquired from a subject by the first system. Moreover, the second sensor data may be a second electrocardiographic waveform acquired from the same subject by the second system.

Further, in this case, the above-described first system may be a system of acquiring an electrocardiographic waveform using at least two electrodes to be assumedly in contact with a subject, and the above-described second system may be a system of acquiring an electrocardiographic waveform using at least three electrodes attached directly on the skin of the subject (three-point inductive method, for example).

For example, when the subject is a driver driving a mobile body such as a vehicle, two electrodes used in the above-described first system may be provided at a seat on which the subject is seated and at a device operated by the subject (a steering, for example).

In the above-described configuration, it is possible to acquire high-accuracy data generated by removing noises occurred due to the movement of a driver's body, vibrations of the vehicle, and the like, while keeping the advantages of the second system such as that the driver is not caused to feel annoyed.

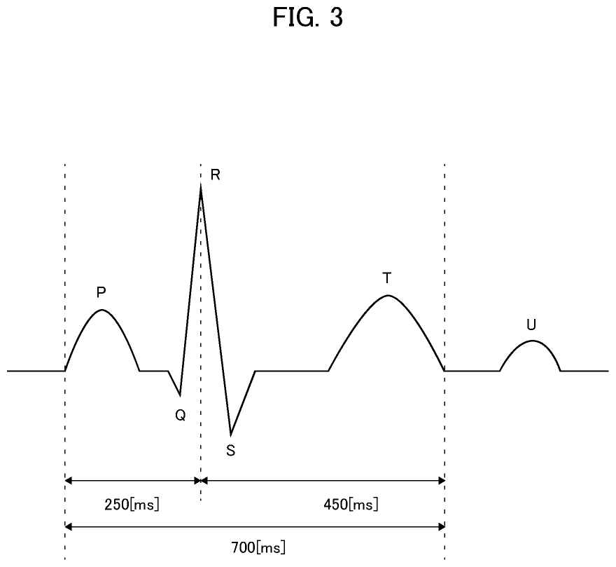

Here, the feature points (feature waveforms) of a general electrocardiographic waveform will be described. FIG. 3 is a diagram illustrating an example of a general electrocardiographic waveform in a single cycle. Note that in FIG. 3, the horizontal axis indicates the lapse of time and the vertical axis indicates a change in voltage. As illustrated in FIG. 3, a plurality of feature waveforms exhibiting characteristic forms can be observed in the general electrocardiographic waveform. The examples of the feature waveform include a P wave, Q wave, R wave, S wave, QRS wave (formed by a Q wave, R wave, and S wave), T wave, U wave, and the like. Moreover, such feature waveforms have the regularity of appearing in the order described above in the time axis.

Among them, the R wave, for example, is an important feature waveform as an index of heartbeat variation (fluctuation). The interval between an R wave in a cycle and an R wave in the following cycle (RRI: R-R Interval) is used to calculate a heartbeat cycle. It is also known that a fluctuation occurs in the RRI due to stress and tiredness, and thus the RRI is an effective physiological index also for detecting a physical burden or mental burden of a subject. In addition, the Q-T interval (QTI) between a Q wave and a T wave in a cycle, for example, indicates time from the start of ventricular excitation to the disappearance of the excitation, and is an important physiological index for detecting an irregular pulse or the like.

In this manner, one cycle of the electrocardiographic waveform includes a plurality of feature waveforms useful for acquiring physiological indices. From this, in the learning according to the embodiment, the entire one cycle may be set as a repetition section, and a feature waveform in accordance with an arbitrary physiological index to be acquired may be set as a feature point.

Meanwhile, one cycle of an electrocardiographic waveform includes therein a section in which the feature waveforms useful for acquiring physiological indices are concentrated. As illustrated in FIG. 3, a P wave, Q wave, R wave, S wave, and T wave can be continuously observed in the time length of around 700 ms. For this reason, in the learning according to the embodiment, the section from the start time point of the P wave to the end time point of the T wave may be set as a repetition section. In this manner, it is possible to learn, with higher accuracy, the regularity in the time axis among the P wave, Q wave, R wave, S wave, and T wave in the repetition section.

Moreover, in the above-described repetition section, the R wave can be observed at the time point of around 250 ms from the start time point of the P wave, for example. From this, in a case where the R wave is set as a target feature point, the learning unit 110 of the embodiment may learn the output related to the R wave, with the use of teacher data based on the second sensor data acquired at a time point when the time length (250 ms) from the start time point of the P wave to a time point at which the R wave is expected to appear has elapsed since the start time point of the time length (700 ms) related to the first sensor data.

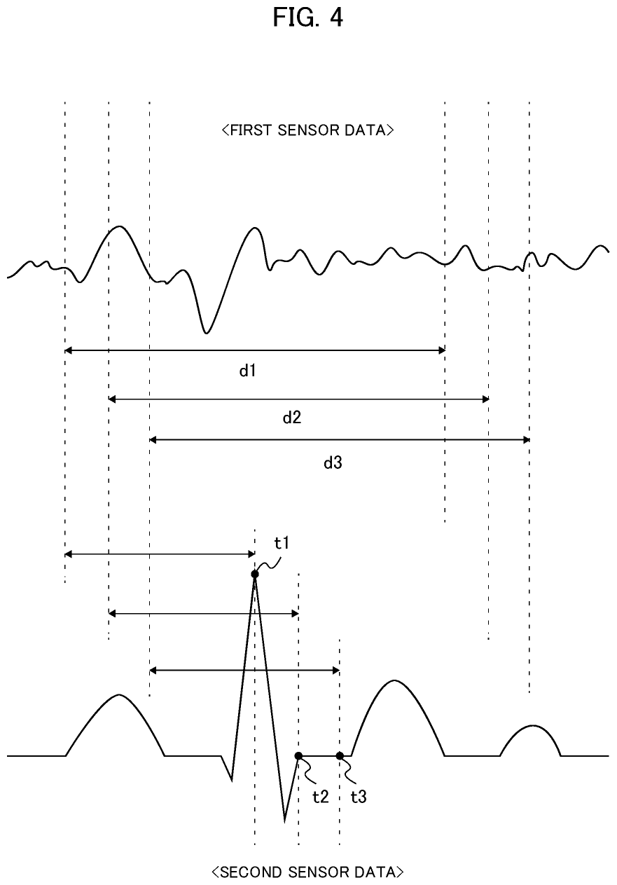

FIG. 4 is a diagram illustrating a correspondence example between learning data and teacher data according to the embodiment. FIG. 4 illustrates, in the upper stage thereof, the first sensor data (first electrocardiographic waveform) acquired from a subject. FIG. 4 also illustrates, in the lower stage thereof, the second sensor data (second electrocardiographic waveform) acquired from the same subject in the same period as the acquisition period of the first sensor data.

In this case, for example, the learning unit 110 may perform learning with the use of the first sensor data acquired in the time length d1 corresponding to the section of 700 ms from the start time point of the P wave to the end time point of the T wave, as the first sequence learning data, and of the second sensor data acquired at the time t1 when 250 ms has elapsed since the start time point of the time length d1, as teacher data.

Similarly, the learning unit 110 may perform learning with the use of the first sensor data acquired in the time length d2 as the second sequence learning data and of the second sensor data acquired at the time t2 when 250 ms has elapsed since the start time point of the time length d2 as teacher data.

Further, similarly, the learning unit 110 may perform learning with the use of the first sensor data acquired in the time length d3 as the third sequence learning data, and of the second sensor data acquired at the time t3 when 250 ms has elapsed since the start time point of the time length d3 as teacher data.

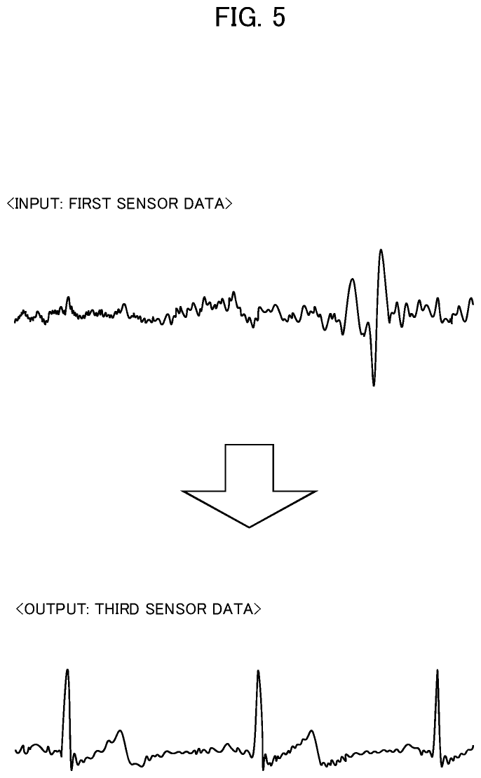

In the above-described data set, it is possible to effectively learn the regularity in the time axis between the R wave and other feature waveforms included in the repetition period. Moreover, the measurement unit 220 of the embodiment can perform the measurement related to the R wave with high accuracy using a learned model constructed by learning with the above-described data set. FIG. 5 is a diagram illustrating an image of the measurement of a target feature point by the measurement unit 220 of the embodiment.

As illustrated in FIG. 5, the measurement unit 220 of the embodiment inputs the first sensor data (first electrocardiographic waveform) in the learned model constructed using the data set exemplified in FIG. 4, whereby it is possible to output the third sensor data (third electrocardiographic waveform) generated by removing noises from the first sensor data. In this manner, even in a case where it is difficult to directly measure the R wave from the first sensor data due to noises, the R wave can be measured with high accuracy on the basis of the third sensor data.

Further, the above has described the case where the learning unit 110 performs learning with the use of the second sensor data itself (a voltage value of the second electrocardiographic waveform, for example) as teacher data. However, the learning unit 110 of the embodiment may learn the output related to the presence probability of a target feature point in the first sensor data with the use of, as teacher data, presence probability data indicating the presence probability of the target feature point in the second sensor data.

For example, in the case of an example illustrated in FIG. 4, the learning unit 110 performs learning regarding the first sensor data (learning data) acquired in the time length d1 with the use of, as teacher data, the presence probability data of an R wave generated on the basis of the second sensor data acquired at the time t1. If the presence probability data represents the presence probability of the R wave by two values of 0 (absent) or 1 (present), the presence probability data of the R wave at the time t1 is 1 because the R wave is present at the time t1. On the other hand, the R wave is absent at the time t2 and the time t3. Therefore, the presence probability data of the R wave at the time t2 and the time t3 is 0.

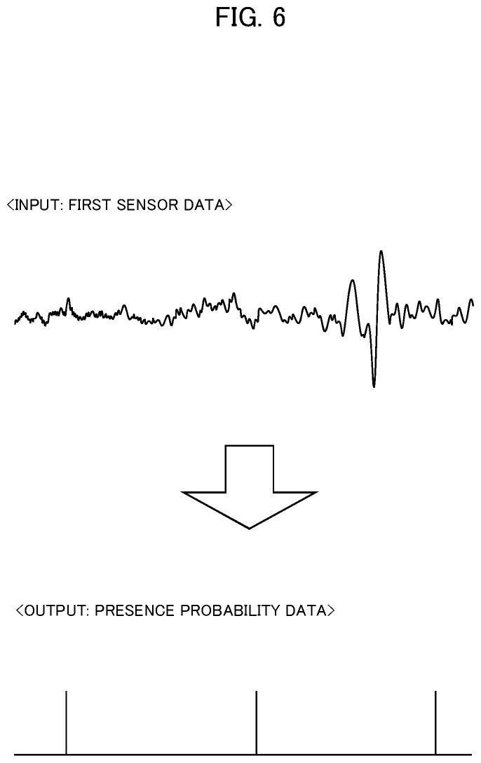

In a case where the learning is performed with the use of the above-described presence probability data as teacher data, the measurement unit 220 of the embodiment inputs the first sensor data (first electrocardiographic waveform) in the learned model, whereby it is possible to directly output the presence probability data of the R wave, as illustrated in FIG. 6. In this manner, in the learning according to the embodiment, the teacher data in accordance with the data format to be output by the measurement unit 220 may be used. Note that although the above has exemplified the case in which the presence probability data is of two values of 0 or 1, the presence probability data may be of three or more values.

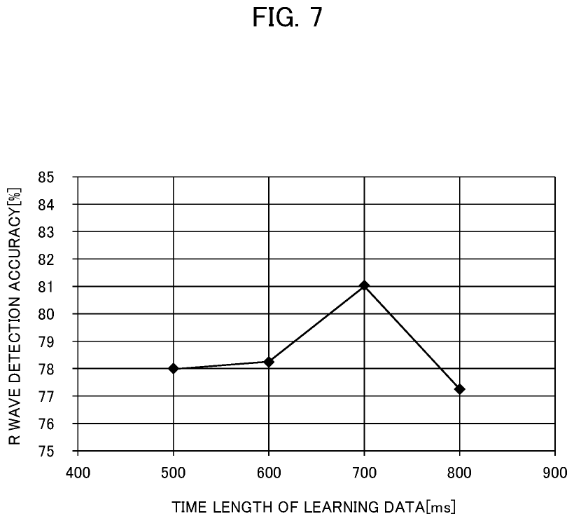

Here, there will be shown a result of the verification regarding the accuracy of R wave detection using the learned model constructed by the learning according to the embodiment. FIG. 7 is a diagram illustrating the accuracy of R wave detection using the learned model of the embodiment. Note that FIG. 7 illustrates the accuracy of R wave detection using the learned models individually constructed with the time length of learning data (first sensor data) set to 500 ms, 600 ms, 700 ms, and 800 ms. Note that in any of the cases, learning was performed with the use of teacher data based on the second sensor data acquired at the time point when 250 ms had elapsed since the start time point of the time length related to the learning data.

As a result, as illustrated in FIG. 7, the learned model constructed by the learning using the learning data of 700 ms was able to detect an R wave with highest accuracy. Such a verification result indicates that more effective learning is performed by setting the time length of learning data in accordance with the regularity in the time axis among a target feature point and other feature points.

Meanwhile, the setting of the time length to 700 ms is merely an example. It is assumed that the optimal time length of learning data is varied on the basis of statistical features of the first sensor data used as learning data. For example, in a case where the average of the time length from the P wave start time point to the T wave end time point is 650 ms in the first sensor data acquired under certain conditions, the time length of learning data may be set to 650 ms. Note that the same applies to the time length of teacher data. For example, in a case where the average of the time length from the P wave start time point to the R wave is 300 ms in the acquired first sensor data and second sensor data, there may be used teacher data based on the second sensor data acquired at a time point when 300 ms has elapsed since the start time point of the time length related to the learning data.

<Flow of Learning Phase and Measurement Phase>

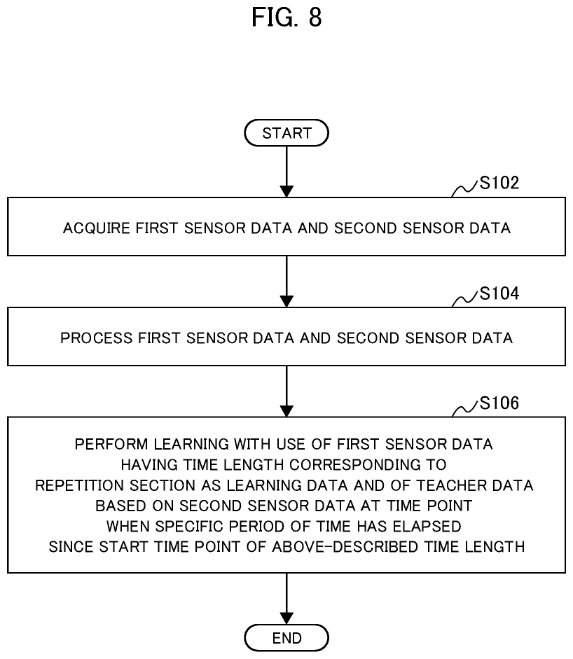

The following will describe flows of the learning phase for learning using the learning device 10 and the measurement phase for measurement using the measurement device 20 according to the embodiment. FIG. 8 is a flowchart illustrating a flow of the learning phase according to the embodiment.

As illustrated in FIG. 8, in the learning phase of the embodiment, the first sensor data and the second sensor data are acquired first (S102). Here, the first sensor data and the second sensor data may be acquired together with the information of time stamps and the like so that the synchronization in the time axis is possible. Moreover, the first sensor data and the second sensor data may be acquired by a separate device from the learning device 10. The acquired first sensor data and second sensor data are stored in the storage unit 120 of the learning device 10.

Next, the first sensor data and the second sensor data are processed if necessary (S104). For example, in a case where the presence probability data related to a target feature point is used as teacher data, the processing of converting the second sensor data acquired at Step S102 into presence probability data may be performed at Step S104. Moreover, various kinds of filter processing for reducing noises in the first sensor data and the second sensor data, or the like may be performed. Note that the above-described processing may be performed by a separate device from the learning device 10.

Next, the learning unit 110 performs learning with the use of the first sensor data having a time length corresponding to the repetition section as learning data and of the teacher data based on the second sensor data acquired at a time point when a specific period of time has elapsed since the start time point of the above-described time length (S106). Here, the learning unit 110 may use the second sensor data itself (or the second sensor data having been subjected to filter processing) as teacher data, or the presence probability data generated at Step S104 as teacher data.

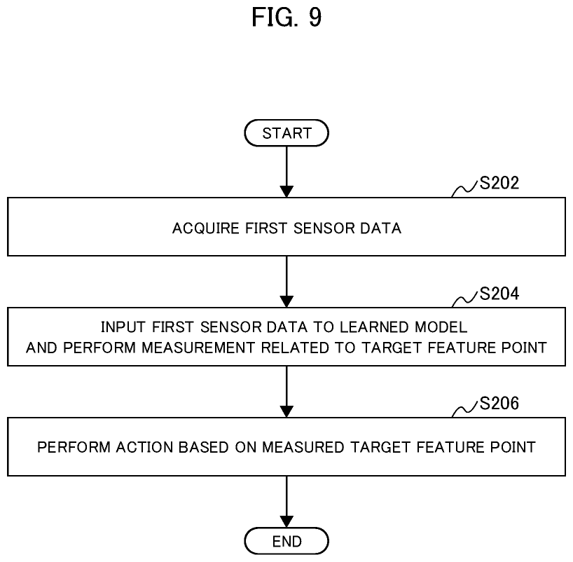

The above has described the flow of the learning phase according to the embodiment. The following will describe a flow of the measurement phase according to the embodiment. FIG. 9 is a flow chart illustrating a flow of the measurement phase according to the embodiment.

As illustrated in FIG. 9, in the measurement phase of the embodiment, the acquisition unit 210 first acquires the first sensor data by the first system (S202). The acquisition unit 210 may acquire, as the first sensor data, an electrocardiographic waveform of a driver using a plurality of electrodes arranged at a steering and a seat of a vehicle, for example.

Next, the measurement unit 220 inputs the first sensor data acquired at Step S202 to a learned model, and performs measurement related to the target feature point included in the first sensor data (S204). In a case where the learning is performed with the use of the second sensor data as teacher data in the learning phase, the measurement unit 220 output the third sensor data generated by removing noises from the first sensor data, and measures the target feature point. Meanwhile, in a case where the learning is performed with the use of the presence probability data as teacher data in the learning phase, the measurement unit 220 outputs presence probability data indicating the presence probability of the target feature point, and measures the target feature point.

Next, various kinds of actions based on the target feature point measured at Step S204 are performed if necessary (S206). For example, in a case where the target feature point is an R wave, the above-described action may be a notification based on an RRI, or the like. The above-described action may be performed by a separate device from the measurement device 20.

<Supplement>

Heretofore, preferred embodiments of the present invention have been described in detail with reference to the appended drawings, but the present invention is not limited thereto. It is obvious that a person skilled in the art can arrive at various alterations and modifications within the scope of the technical ideas defined in the claims, and it should be naturally understood that such alterations and modifications are also encompassed by the technical scope of the present invention.

For example, the above-described embodiment has exemplified, as a main example, the case in which the learning unit 110 learns the measurement related to the cardiac activity of a subject. However, the object to be learned by the learning unit 110 is not limited to the measurement of vital data as described above. The learning unit 110 is also able to measure various kinds of data indicating an operation state of an arbitrary device, for example.

Moreover, the above-described embodiment has exemplified, as the first system of acquiring an electrocardiographic waveform, the system in which electrodes are arranged at positions to be assumedly in contact with a subject, and has exemplified, as the second system, the system in which electrodes are attached directly on the skin of a subject. However, the first system and the second system in the present technology may be arbitrary different systems having a difference therebetween in susceptibility to influences by noises. For example, in the case of acquiring a heartbeat, the first system may be a non-contact system using a doppler sensor. In this case, the second system may be an arbitrary system less affected by noises than such a non-contact system. For example, the second system in such a case may be the above-described contact system in which electrodes are attached on the skin of a subject. In this manner, the first system of the present technique is not limited to the system exemplified in the above-described embodiments, and may be selected appropriately. Furthermore, in a case where the contact system of acquiring an electrocardiographic waveform using at least two electrodes to be assumedly in contact with a subject is less affected by noises than the non-contact system using a doppler sensor or the like, the non-contact system may be the first system, and the contact system may be the second system.

A sequence of processing by the devices described in this specification may be achieved using any one of software, hardware, and the combination of software and hardware. A program forming the software is preliminarily stored in, for example, a recording medium (non-transitory media) provided inside or outside each device. Then, each program is read in a random access memory (RAM) when executed by a computer, and executed by a processor such as a central processing unit (CPU). The above-described recording medium is, for example, a magnetic disk, an optical disk, a magneto-optical disk, a flash memory, or the like. Moreover, the above-described computer program may be distributed through a network, for example, without using any recording medium.

### REFERENCE SIGNS LIST

10 learning device
110 learning unit
120 storage unit
20 measurement device
210 acquisition unit
220 measurement unit

1. A learning device, comprising:
a learning unit that learns output related to a target feature point to be observed in a repetition section observed periodically along a progress of time, with the use of first sensor data being acquired by a first system and having a time length corresponding to the repetition section, as learning data, and
of teacher data based on second sensor data acquired by a second system at a time point when a specific period of time has elapsed since a start time point of the time length related to the first sensor data, the second system being less affected by noises than the first system, wherein
the specific period of time is set on the basis of a time length from a start time point of the repetition section to a time point at which the target feature point is expected to appear.

2. The learning device according to claim 1, wherein the repetition section includes at least another feature point having a regularity, regarding an appearance of the another feature point, in a time axis with the target feature point.

3. The learning device according to claim 1, wherein the first sensor data and the second sensor data are electrocardiographic waveforms recording a cardiac activity of a subject.

4. The learning device according to claim 3, wherein the repetition section is a section from a start time point of a P wave to an end time point of a T wave.

5. The learning device according to claim 4, wherein
the target feature point is an R wave, and
the learning unit learns output related to an R wave with the use of teacher data based on the second sensor data acquired at a time point when a time length from a start time point of a P wave to a time point at which an R wave is expected to appear has elapsed since a start time point of the time length related to the first sensor data.

6. The learning device according to claim 5, wherein the learning unit learns output related to a presence probability of an R wave in the first sensor data with the use of presence probability data indicating a presence probability of an R wave in the second sensor data, as teacher data.

7. The learning device according to claim 3, wherein
the first system is a system of acquiring an electrocardiographic waveform using at least two electrodes to be assumedly in contact with the subject, and
the second system is a system of acquiring an electrocardiographic waveform using at least three electrodes attached on a skin of the subject.

8. The learning device according to claim 3, wherein the subject is a driver driving a mobile body.

9. A learning method, comprising:
learning output related to a target feature point to be observed in a repetition section observed periodically along a progress of time, with the use of first sensor data being acquired by a first system and having a time length corresponding to the repetition section, as learning data, and
of teacher data based on second sensor data acquired by a second system at a time point when a specific period of time has elapsed since a start time point of the time length related to the first sensor data, the second system being less affected by noises than the first system, wherein
the specific period of time is set on the basis of a time length from a start time point of the repetition section to a time point at which the target feature point is expected to appear.

10. A measurement device, comprising:
a measurement unit that performs measurement related to a target feature point to be observed in first sensor data, with the first sensor data acquired by a first system as an input, wherein
the measurement unit performs measurement related to the target feature point using a learned model constructed by learning output related to the target feature point in a repetition section observed periodically along a progress of time with the use of the first sensor data having a time length corresponding to the repetition section, as learning data, and of teacher data based on second sensor data acquired by a second system at a time point when a specific period of time has elapsed since a start time point of the time length related to the first sensor data, the second system being less affected by noises than the first system, and
the specific period of time is set on the basis of a time length from a start time point of the repetition section to a time point at which the target feature point is expected to appear.
